(19) United States
(12) Patent Application Publication
(10) Pub. No.: US 2004/0230121 A1
(43) Pub. Date: Nov. 18, 2004

(54) Ultrasonic contrast agent imaging by dualband pulse transmission

(76) Inventors: Rune Hansen, Karli (NO); Bjorn A.J. Angelsen, Trondheim (NO)

Correspondence Address: Lance J. Lieberman, Esq., Cohen, Pontani, Lieberman & Pavane, Suite 1210, 551 Fifth Avenue, New York, NY, 10176, US

(21) Appl. No.: 10/783,121

(22) Filed: Feb. 20, 2004

Related U.S. Application Data
(60) Provisional application No. 60/449,222, filed on Feb. 20, 2003.

(51) Int. Cl. A61B 8/06

(52) U.S. Cl. 600/458

(57) ABSTRACT

A method for improved imaging of ultrasound contrast agent using dual frequency band transmitted pulses is described. The method is based on transmitting a dualband pulse consisting of two frequency bands, preferably a fundamental band and its second harmonic band. In addition, a general pulse inversion method is applied to reduce or cancel first order nonlinear harmonic tissue signal components introduced due to the transmission of dualband pulses.

### CROSS REFERENCE TO RELATED APPLICATION

This application discloses and claims priority from U.S. Provisional Patent Application Ser. No. 60/449,222, filed on Feb. 20, 2003.

### BACKGROUND OF THE INVENTION

1. Field of the Invention

This invention relates to methods and systems for ultrasonic detection and imaging of contrast agents located in soft tissue or tissue fluids.

2. Description of the Related Art

Ultrasound contrast agents are typically made as solutions of micro gas bubbles or nano lipid particles. The gas bubbles typically show strong and nonlinear scattering of the ultrasound, a phenomenon that is used to differentiate the contrast agent signal from the tissue signal. In the earliest applications (˜1985) the increased scattering from the contrast agent within the transmitted frequency band was used to enhance the scattering from blood. Later, second harmonic components in the nonlinearly scattered signal were used to further enhance the contrast agent signal above the tissue signal in methods generally referred to as nonlinear contrast harmonic imaging.

The following two signal power ratios have vital importance for the quality of performance of a contrast imaging system:

CTR—Contrast signal to Tissue signal Ratio. This gives the ratio of the signal power scattered from the contrast agent in a region to the signal power scattered from the tissue in that region. This ratio is often referred to as specificity.

CNR—Contrast signal to Noise Ratio. This gives the ratio of the signal power scattered from the contrast agent in a region to the noise power in that region. This ratio is often referred to as sensitivity.

The CNR determines the maximum depth for imaging the contrast agent while the CTR describes the enhancement of the contrast agent signal above the tissue signal in the image and thus the capability of differentiating contrast signal from tissue signal. High values of both these ratios are therefore necessary for good imaging of the contrast agent.

The nonlinear distortion of the signal scattered from the contrast agent is much stronger than for the tissue signal, a phenomenon that is extensively used to enhance the CTR. In one method, received tissue signal components in the transmitted frequency band (linear components) are reduced by combining the received signal from two transmitted pulses with different amplitudes. In other methods, the second harmonic band of the nonlinearly scattered signal is obtained either by bandpass filtering or by combining the received signals from two or more transmitted pulses with different polarities.

Wave propagation in soft tissue is a weak nonlinear process for intensities common in medical ultrasound imaging. The local nonlinearity is low and the distortion of the wave accumulates gradually in the propagation direction of the wave. Wave propagation in a homogeneous medium with acoustic absorption may be mathematically described by the following equation

$${∇}^{2} p - \frac{1}{{c}^{2}} \frac{{\partial }^{2} p}{\partial {t}^{2}} - \frac{1}{{c}^{2}} h ⊗ \frac{{\partial }^{2} p}{\partial {t}^{2}} = - κ β \frac{1}{{c}^{2}} \frac{{\partial }^{2} {p}^{2}}{\partial {t}^{2}} \qquad ( 1 )$$

where p is the acoustic pressure, c is the speed of sound, ∇² is the Laplacian sum of the second derivatives with respect to the three Cartesian coordinates, h is a causal filter, κ is the compressibility, β is a nonlinearity parameter for the propagation medium, and t is time. The integral term is responsible for acoustic absorption while the term on the right-hand side is responsible for nonlinear effects in the wave propagation.

In medical ultrasound imaging, a good approximation of the transmitted field is often to first calculate the linear transmitted field by discarding the term on the right-hand side in the above equation and to use this linear field as a source for the second harmonic component which then is calculated by using the full equation. This is usually referred to as the quasi-linear approximation of the nonlinear wave equation and the nonlinear component is then produced by a quadratic effect of the linear transmitted field only.

This is a first approximation of the nonlinearity of the propagation medium but since wave propagation in absorbing soft tissue at intensities common in medical ultrasound imaging is a relatively weak nonlinear process, this approximation is good.

From a homogeneous medium described by this equation there will be no back-scattered waves. In an actual medical ultrasound imaging situation, tissue heterogeneities are responsible for producing the back-scattered waves and hence the image displayed on the ultrasound scanner. In numerical computer simulations, a simulated scatterer can be inserted at a desired location in the calculated transmit field to produce a back-scattered wave. The scattering process can then, as in the case of the heterogeneous tissue, usually be modeled as a linear process. In the case of the point scatterer, the scattered amplitude is proportional to the square of the frequency.

Ultrasound contrast agents, and in particular gas filled bubbles, generally exhibit a strong nonlinear scattering when subject to an imaging ultrasound pulse, and higher order nonlinear terms are not negligible. A well known nonlinear equation for the motion of the fluid around a spherical pulsating gas bubble is the Rayleigh-Plesset equation. The radius oscillation of the bubble is then expressed as

$$ρ ( a \ddot{a} + \frac{3}{2} {\dot{a}}^{2} ) = p ( a , t ) - {p}_{0} - {p}_{i} ( t ) \qquad ( 2 )$$

where a is the radius, \dot{a} and ä is the velocity and acceleration of the bubble radius, ρ is the density of the surrounding fluid, p₀ is the ambient equilibrium pressure, pᵢ(t) is the driving pressure, and p(a,t) is the pressure at the bubble surface. Nonlinear effects of the gas and encapsulating thin shell, surface tension, and viscosity may be incorporated in the first term on the right-hand side of Eq.2.

### SUMMARY OF THE INVENTION

To increase the nonlinear fundamental and/or second and/or third and/or fourth harmonic CNR, pulses with frequency components in two frequency bands, in particular a fundamental band and its second harmonic band, are transmitted. With stationary tissue, resulting linear and first order nonlinear harmonic tissue components can be significantly reduced or canceled by a pulse inversion technique, where for example two multiband pulses with opposite polarities on the second harmonic band are consecutively transmitted and the received signals from these two pulses are combined through summation or subtraction. With nonstationary tissue, more than two transmit pulses may be used to account for the tissue movement between each pulse.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

The decibel plots of scattered pressure from a contrast bubble and transmit pressure pulses should not be compared in terms of absolute levels of harmonic components. It is the relative levels of harmonic components which is visualized in these figures.

FIG. 1 shows an example of a fundamental (solid line) and second harmonic (dashed line) pulse. In the upper panel, the phase angle of the second harmonic component relative to the fundamental components is 0 while in the lower panel, this phase angle is set to π/2.

FIG. 2 illustrates four possible dual frequency band pulse designs using a relative phase angle of 0 and π/2 in the upper and lower panels, respectively.

FIG. 3 shows the spectrum of the pulses in FIG. 2.

FIG. 4 depicts the spectrum of the resulting transmit pulse at 6 cm on the symmetry axis of an annular transducer of radius 1 cm and focus at 12 cm when driven by pulse 103 in FIG. 2.

FIG. 5 shows the spectrum of the resulting pulse summation transmit signal at 6 cm on the symmetry axis obtained using the drive pulses 103 and 104 in FIG. 2 to drive the annular transducer.

FIG. 6 shows the spectrum of the resulting pulse subtraction transmit signal at 6 cm on the symmetry axis obtained using the drive pulses 103 and 104 in FIG. 2 to drive the transducer.

FIG. 7 depicts transmit field at 6 cm on the symmetry axis obtained using the fundamental frequency band only in FIG. 3 to drive the annular transducer.

FIG. 8 displays the spectrum of a scattered bubble signal obtained with the transmit field at 6 cm on the symmetry axis using the fundamental frequency band only in FIG. 3 to drive the annular transducer.

FIG. 9 depicts the spectrum of a scattered bubble signal obtained with the transmit field at 6 cm on the symmetry axis using pulse 104 in FIG. 2 to drive the annular transducer.

FIG. 10 depicts the spectrum of a scattered bubble signal obtained with the transmit field at 6 cm on the symmetry axis using pulse 103 in FIG. 2 to drive the annular transducer.

FIG. 11. shows the spectrum of the resulting pulse summation bubble signal at 6 cm on the symmetry axis obtained using the drive pulses 101 and 102 in FIG. 2 on the transducer.

FIG. 12 shows the spectrum of the resulting pulse subtraction bubble signal at 6 cm on the symmetry axis obtained using the drive pulses 103 and 104 in FIG. 2 on the transducer.

FIG. 13 displays the resulting transmit pulse in the time domain at 10 cm on the symmetry axis using pulse 102 in FIG. 2 on the annular transducer.

FIG. 14 displays the resulting transmit pulse in the time domain at 10 cm on the symmetry axis using pulse 101 in FIG. 2 on the annular transducer.

FIG. 15 displays the spectrum of the resulting transmit pulse at 10 cm on the symmetry axis using pulse 101 in FIG. 2 on the annular transducer.

FIG. 16 shows the spectrum of the resulting pulse summation transmit signal at 10 cm on the symmetry axis obtained using the drive pulses 101 and 102 in FIG. 2 on the transducer.

FIG. 17 shows the spectrum of the resulting pulse subtraction transmit signal at 10 cm on the symmetry axis obtained using the drive pulses 101 and 102 in FIG. 2 on the transducer.

FIG. 18 depicts the spectrum of a scattered bubble signal obtained with the transmit field at 10 cm on the symmetry axis using the fundamental frequency band only in FIG. 3 to drive the annular transducer.

FIG. 19 depicts the spectrum of a scattered bubble signal obtained with the transmit field at 10 cm on the symmetry axis using pulse 101 in FIG. 2 to drive the annular transducer.

FIG. 20 shows the spectrum of the resulting pulse subtraction bubble signal at 10 cm on the symmetry axis obtained using the drive pulses 101 and 102 in FIG. 2 on the transducer.

### DETAILED DESCRIPTION OF THE PRESENTLY PREFERRED EMBODIMENTS

A first aspect of the present invention is performed by transmitting a pulse containing two frequency bands, in particular a fundamental band and its second harmonic band where the relative phase relations between the two transmitted frequency bands are adjustable, into an area containing contrast agent. If the intensity of the transmitted pulse is relatively low, the propagation medium without contrast agent will behave approximately linearly while the contrast agent will behave strongly nonlinearly. The area containing contrast agent will thus scatter frequency components not transmitted, in particular third and fourth harmonic components, which can be used for image reconstruction of the area of interest. By using a dual frequency band pulse with adjustable relative phase angle between the two transmitted frequency bands, a large flexibility with respect to transmit pulse design is allowed.

If the transmit fundamental frequency band is chosen such that the resulting third or fourth harmonic component is in the vicinity of the contrast bubble resonance frequency, the bubble will respond with strong third and/or fourth harmonic components when driven by the low intensity dual frequency band pulse. Properly selecting the relative phase angle between the transmitted fundamental and second harmonic band one is able to manipulate and optimize the third and/or fourth harmonic bubble response.

In a second aspect of the present invention, a pulse similar to the one in the first aspect is transmitted, scattered, received, and stored. Then a second pulse is transmitted down the same line of sight where the polarity of the second harmonic frequency band is inverted relative to the first transmitted pulse and a general form of pulse inversion is performed on the resulting received signals. The two transmitted pulses may for example be expressed as

p₁(t)=α₁(t)sin(ωt)+α₂(t)sin(2ωt−φ₁) (3)

p₂(t)=α₃(t)sin(ωt)−α₄ (t)sin(2ωt−φ₂) (4)

where αₙ(t) are positive amplitude functions, ω is the angular frequency, and φₙ are arbitrary phase angles. These phase angles are in the present invention given as a fraction of the temporal period of the second harmonic component. We define the indicated phase angle to zero when the zero-crossings of the fundamental pressure component coincide with every second zero-crossing of the second harmonic pressure component. In the present invention, this phase angle will typically be between 0 and π/2. The fact that α₃ may be different from α₁ and α₄ may be different from α₂ gives the flexibility to further utilize the assumption of strong versus weak nonlinearity of a propagation medium containing and not containing contrast agent, respectively.

If α₃=α₁, α₄=α₂ and φ₂=φ₁ in Eqs.3 and 4, performing pulse summation on two resulting received signals, s₁ and s₂, from a region of tissue not containing contrast agents so that the nonlinearity is well described by the quasi-linear approximation, gives

s₁(t)+s₂(t)=L(2α₁(t)sin(ωt)+2α₁²(t)sin²(ωt)+2α₂²(t)sin²(2ωt−φ₁)) (5)

where L is a linear operator. The first term on the right-hand side is the linear component in the summed signal. The linear component of the transmitted second harmonic component is canceled in the pulse inversion technique due to the phase inversion in Eqs.3 and 4. In the two last terms on the right-hand side in Eq.5 we have the first order nonlinear components in the summed signal. These nonlinear terms consist of a second harmonic component from the transmitted fundamental component, and a second harmonic component from the transmitted second harmonic component (i.e. a fourth harmonic component relative to the transmitted fundamental band). The nonlinear sum and difference frequencies producing third and fundamental harmonic components have hence been canceled in the pulse inversion process for a propagation medium of weak nonlinearity described by the quasi-linear approximation.

If we instead subtract the two received signals, s₁ and s₂, from a region of tissue not containing contrast agents, and where nonlinearities hence are assumed to be described by the quasi-linear approximation, we obtain

s₁(t)−s₂(t)=L(2α₂(t)sin(2ωt−φ₁)+4α₁(t)α₂(t)sin(ωt)sin(2ωt−φ₁)) (6)

Now, the linear transmitted fundamental band is canceled in the pulse inversion process while the linear transmitted second harmonic band is doubled. The first order nonlinear term, second term on the right-hand side in Eq.6, now consists of a nonlinear mixing term between the linear transmitted fundamental band and the second harmonic band producing nonlinear fundamental and third harmonic components. Performing a subtraction instead of a summation of the received scattered signals hence cancels the first order nonlinear fourth harmonic components from the tissue.

Nonlinear scattering from contrast bubbles will not be adequately described by the quasi-linear approximation. Third and fourth harmonic bubble signals will thus not be significantly reduced when performing the indicated pulse summation and pulse subtraction process, respectively. Also, a very strong nonlinear fundamental contrast component can be obtained with the pulse subtraction technique.

In a third aspect of the invention a sequence of pulses as in the second aspect is transmitted down each line of sight. Every second transmitted pulse has inverted polarity on its second harmonic band relative to the previous one as explained above. Only a couple of the transmitted pulses may be used for image reconstruction which is done performing a general form of pulse inversion or applying standard Doppler processing techniques. Transmitting a sequence of pulses like this instead of only two pulses may be advantageous in relation to moving tissue problems encountered in pulse inversion contrast techniques.

In the second and third aspect of the present invention, two or more pulses are to be transmitted down each line of sight, i.e. in one beam direction. If the image is formed by stepping the beam direction, for example as with electronic scanning, the beam direction can be the same for as long as necessary and several consecutive pulses can be transmitted down the same line of sight.

The image may also be formed using a beam that is continuously swept with a mechanical motion of the transducer. It is then important that this sweeping motion is slow in relation to the pulse transmit rate so that the received signals from the transmitted pulses being combined to detect the contrast agent signal originate from substantially the same region in the tissue.

The subsequent received signals can be combined through simple summation or subtraction, or the received signals may be linearly combined.

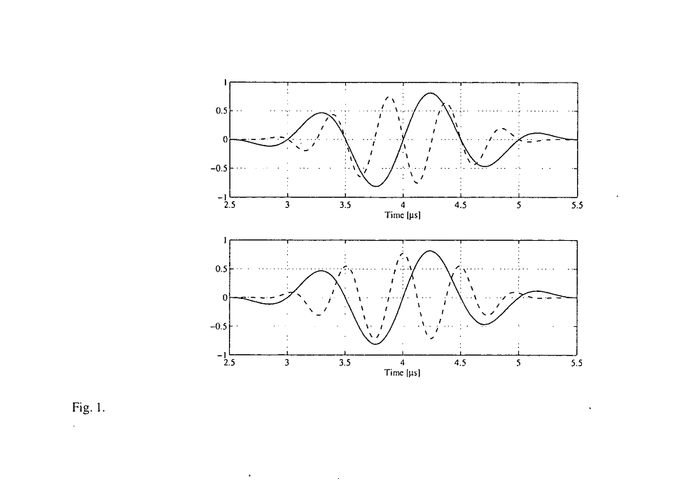

FIG. 1 shows an example of a fundamental (solid line) and second harmonic (dashed line) pulse. In the upper panel, the phase angle of the second harmonic component relative to the fundamental components is 0 while in the lower panel, this phase angle is set to π/2.

As already indicated, transmitting dual frequency band pulses with adjustable relative phase relations (selecting φ) gives a large flexibility with respect to transmit pulse design. The pulses 101, 102, 103, and 104 depicted in FIG. 2 show examples of four possible transmit pulse designs. Pulse 101 is described by Eq.3 where α₂=0.8α₁ and φ₁=0 while pulse 102 is described by Eq.4 where α₃=α₁, α₄=α₂, and φ₂=φ₁. Similarly, pulse 103 is described by Eq.3 where α₂=0.8α₁ and φ₁=or π2 while pulse 104 is described by Eq.4 where α₃=α₁, α₄=α₂, and φ₂=φ₁. Comparing pulse 101 and 102 there is a small difference in terms of maximum amplitude levels due to the phase inversion of the second harmonic band whereas in pulse 103 and 104 there is a major asymmetry in terms of positive and negative amplitude levels.

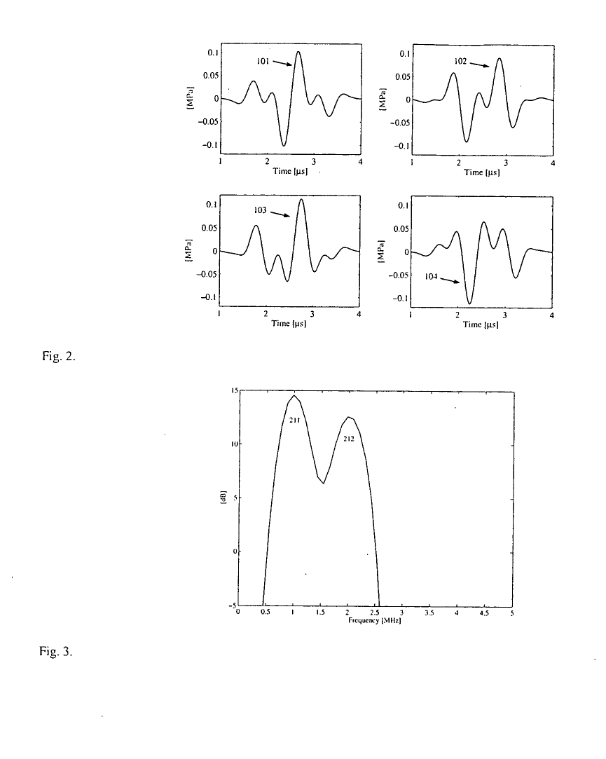

The absolute value of the Fourier Transform will be the same for the four pulses in FIG. 2 and is shown in FIG. 3. We see that the pulses contain a fundamental 1 MHz component (211) and a second harmonic 2 MHz component (212).

The pulses in FIG. 2 are then used as excitation pulses on an annular transducer with radius equal to 1 cm and a geometrical focus at 12 cm. A numerical simulation program developed in our group is then used to calculate the transmitted acoustical fields from this annular transducer when driven by the excitation pulses consisting of both a fundamental band (211) and a second harmonic band (212). The simulation program includes effects of diffraction, frequency dependent absorption, and nonlinear tissue elasticity. Effects of nonlinear tissue elasticity are not limited by the quasi-linear approximation in the simulation program.

FIG. 4 shows the spectrum of the transmit field at 6 cm on the symmetry axis when pulse 103 is applied on the annular transducer. There is a strong linear fundamental (361) and linear second harmonic (362) band and weaker nonlinear third (363) and fourth (364) harmonic bands introduced due to nonlinear tissue elasticity.

We may now perform a pulse summation process by adding the transmit fields obtained by exciting the annular transducer with pulse 103 and 104, respectively. Tissue scattering and back-propagation of the scattered pulses are assumed to be linear processes. Performing pulse inversion on the obtained transmit fields thus give a good indication on how the harmonic pulse inversion process will affect the received tissue signals. FIG. 5 displays the spectrum of the resulting pulse summation signal at 6 cm on the symmetry axis. As expected from Eq.5, the fundamental component (371) is increased by around 6 dB relative to FIG. 4 while the linear transmitted second harmonic band is canceled and only a nonlinear component (372) remains. The third harmonic band (373) is in the pulse summation process significantly reduced while the fourth harmonic band (374) shows an expected increase relative to FIG. 4.

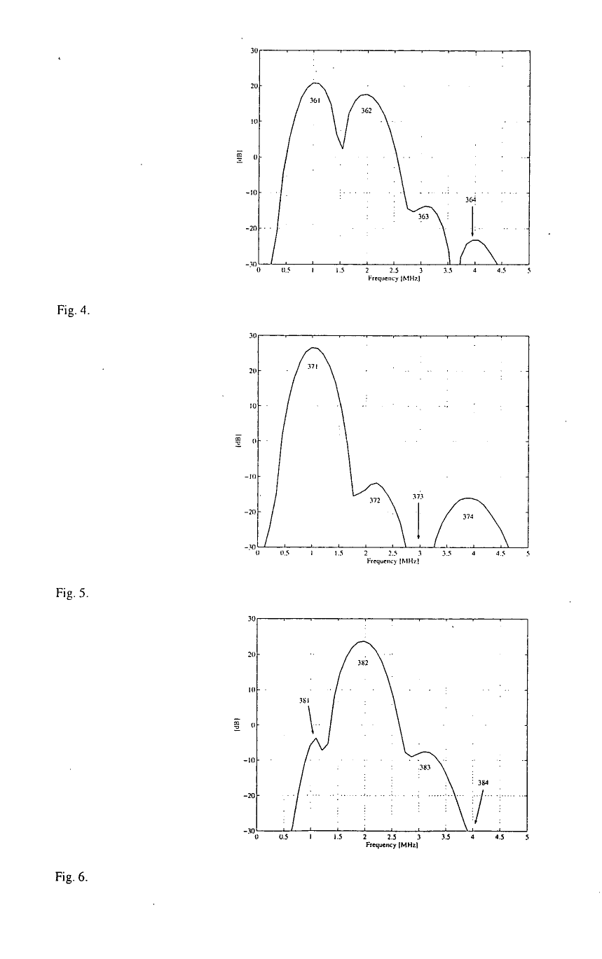

Performing a pulse subtraction of the two transmit fields obtained from pulse 103 and 104 we obtain the spectrum depicted in FIG. 6 at 6 cm on the symmetry axis. The linear fundamental band is now canceled and only a nonlinear component (381) can be seen. The linear second harmonic band (382) and nonlinear third harmonic band (383) are both increased by approximately 6 dB while the fourth harmonic band (384) is heavily suppressed relative to FIG. 4. These results are in good agreement with Eq.6.

By removing the second harmonic band from the pulses in FIG. 2 they will all be identical. The transmit field from such a conventional single frequency band pulse is calculated. At 6 cm on the symmetry axis the spectrum of the transmit field is as shown in where a strong linear fundamental band (391) and a weak first order nonlinear band (392) are seen.

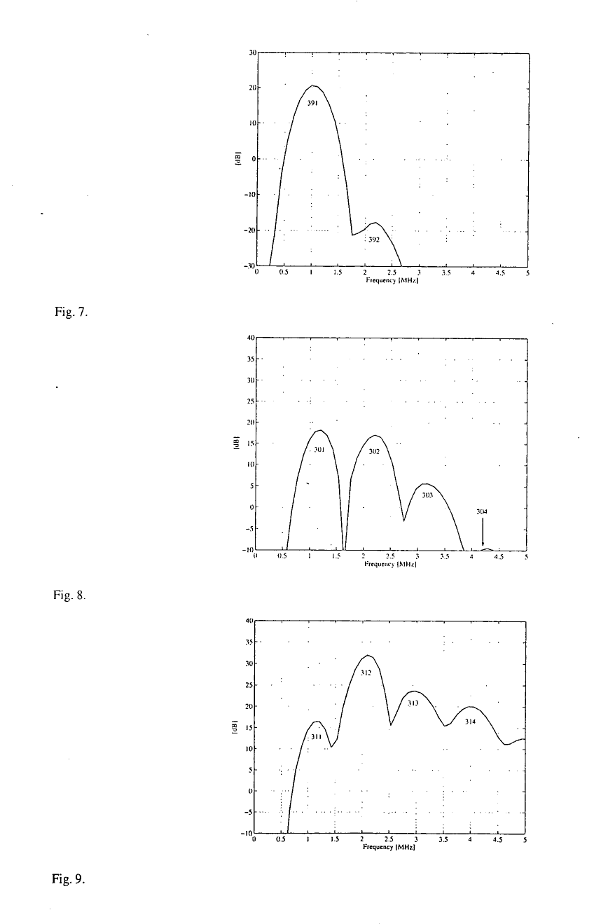

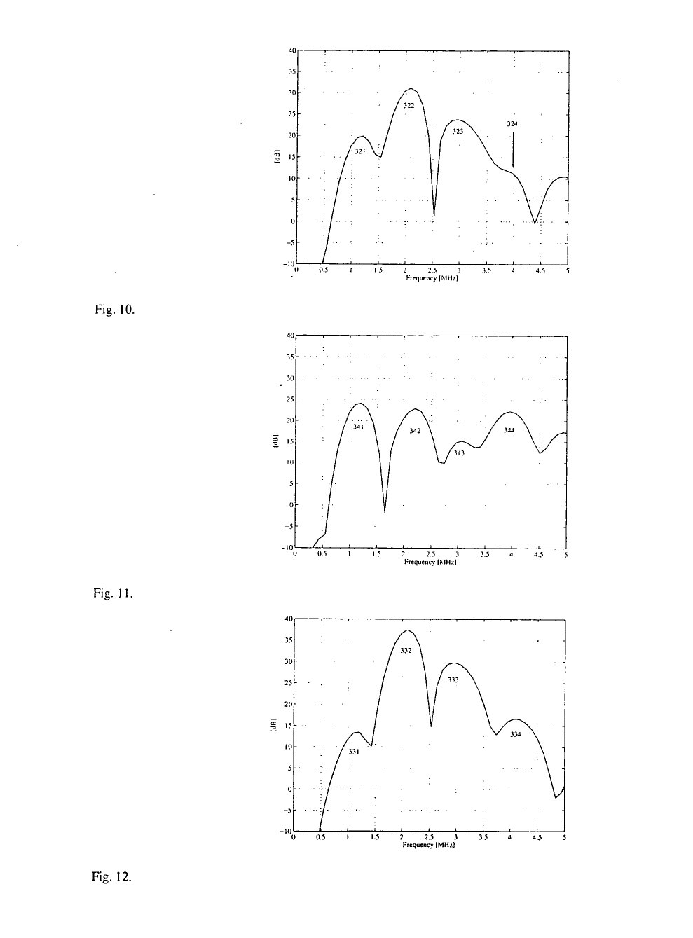

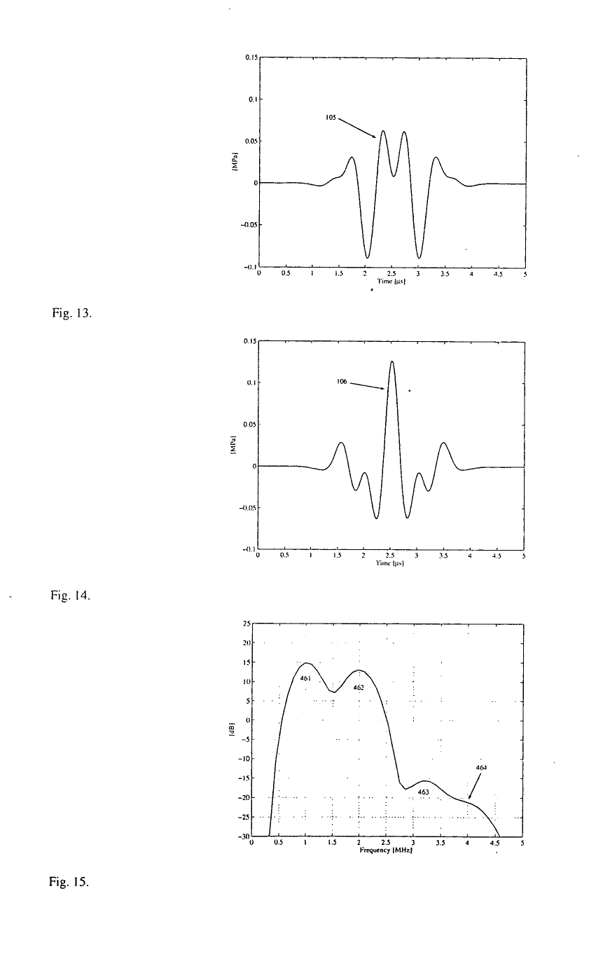

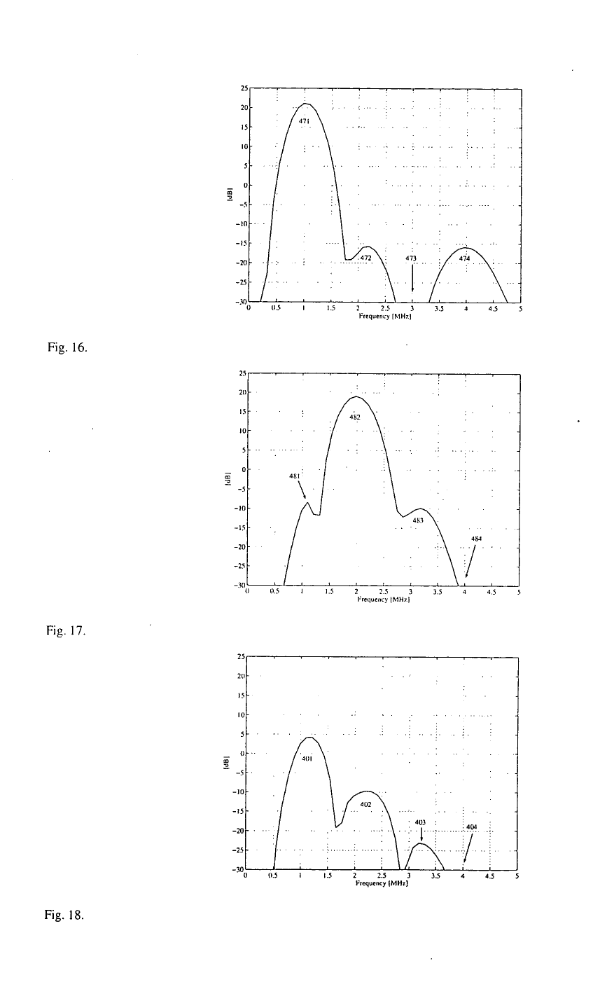

The transmit field from such a single frequency band pulse is used to drive a contrast bubble with resonance frequency around 3 MHz. The spectrum of the scattered bubble signal when applying the resulting transmit field at 6 cm on the symmetry axis as drive pulse is displayed in FIG. 8. There is a strong scattered linear component (301) and first order nonlinear second harmonic component (302). Higher order nonlinear scattered components, third (303) and especially fourth (304) harmonic components, are weak.

Driving the same bubble with the transmit fields obtained from the dual frequency band pulses (104 and 103) we obtain the scattered spectra displayed in FIG. 9 and FIG. 10 at 6 cm on the symmetry axis. Due to the linear transmitted second harmonic component, the scattered second harmonic from the bubble (312 and 322) is increased. The scattered third (313 and 323) and fourth (314 and 324) harmonic bands are now mainly first order nonlinear terms and significantly increased relative to the situation applying a single frequency band pulse in FIG. 8. Also important, we notice that there is a difference in scattered fundamental bubble signal (compare 311 and 321) and scattered fourth harmonic bubble signal (compare 314 and 324) applying the two pulses 104 and 103 with inverted polarity on the transmitted second harmonic band.

Performing pulse summation on the two scattered bubble signals driven by the dual frequency band transmit fields at 6 cm we obtain the spectrum depicted in FIG. 11. Most important here is that the resulting third harmonic component (343), although somewhat reduced relative to 313 and 323, is increased relative to 303.

We may also do a pulse subtraction of the two scattered bubble signals driven by the dual frequency band transmit fields at 6 cm and the resulting spectrum is shown in FIG. 12. When performing pulse subtraction we are especially interested in the resulting fundamental and fourth harmonic bands from the contrast bubble. Energy at these bands were in FIG. 6 seen to be very low for tissue (381 and 384). The pulse subtraction bubble signal, however, has a strong nonlinear fundamental component (331) only reduced by a few decibels compared to 311 in FIG. 9. Transmitting dual frequency band pulses and doing pulse inversion on the transmitted second harmonic band hence appears to be a very good alternative to contrast amplitude modulation technique with nonlinear fundamental imaging. The resulting fourth harmonic contrast component (334) is also very strong and can be the basis for image reconstruction.

FIG. 13 displays the transmit pulse in the time domain at 10 cm on the symmetry axis resulting from driving the annular transducer with pulse 102 while FIG. 14 displays the transmit pulse at the same location obtained with pulse 101. We notice the asymmetry with respect to positive and negative pressure amplitude when using the two excitation pulses with inverted polarity on the transmitted second harmonic band.

In FIG. 15 the spectrum of pulse 105 is shown. In addition to the two strong linear components (461 and 462) a third harmonic band (463) and a fourth harmonic band (464) are present.

In the same way as was done at 6 cm we now perform pulse summation and pulse subtraction of the two transmit fields obtained from pulse 101 and 102 and the resulting spectra are shown in FIG. 16 and FIG. 17. As previously, when doing pulse summation we heavily suppress the second (472) and third (473) harmonic tissue components while the fundamental (481) and fourth (484) harmonic tissue components are suppressed when doing pulse subtraction.

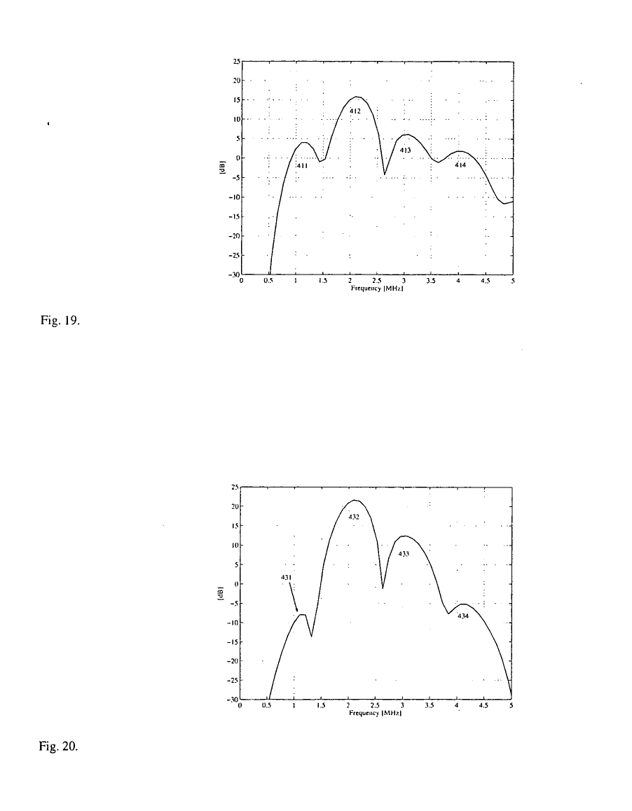

A contrast bubble with resonance frequency around 3 MHz is then driven by the calculated transmit field at 10 cm on the symmetry axis from the fundamental band only of pulse 102. The spectrum of the resulting scattered bubble signal is depicted in FIG. 18 and the bubble response is seen to be rather linear when driven by this low intensity single frequency band pulse.

The bubble is then driven by the calculated transmit field at 10 cm on the symmetry axis from pulse 102. The spectrum of the resulting scattered bubble signal is depicted in FIG. 19. Due to strong nonlinear scattering from the bubble, the third (413) and fourth (414) harmonic components are very high compared to 403 and 404 obtained using a single frequency band pulse.

In FIG. 20 the spectrum of the resulting pulse subtraction signal at 10 cm on the symmetry axis is seen for the situation when pulse 101 and 102 are used to drive the annular transducer. Again, we see a strong nonlinear fundamental band (431) and a strong fourth harmonic band (434) which can be used for image reconstruction of the contrast bubble since these components are heavily suppressed for tissue (481 and 484).

The new contrast specific imaging method presented is capable of significantly increasing the first order nonlinear signal scattered from contrast agents without increasing the resulting first order nonlinear signal scattered from tissue. This new method will thus give rise to detection of contrast agents in the presence of tissue with better sensitivity (CNR) and specificity (CTR).

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

We claim:

1. A method for imaging of ultrasound contrast agents in a region of tissue, where
an ultrasound pulse containing frequencies in a first harmonic band and the second harmonic component of this band is transmitted towards said region,
where the phase of said second harmonic band relative to said first harmonic band is adjusted between 0 and
$$\frac{π}{2}$$
for maximal received nonlinear third and/or fourth harmonic components in the received signals from the contrast agent.

2. A method for imaging of ultrasound contrast agent according to claim 1, where RF-filtering is applied on the received signals to attenuate unwanted tissue components.

3. A method for imaging of ultrasound contrast agents in a region of tissue, where
at least two ultrasound pulses are transmitted consecutively towards said region with substantially the same focus and direction of the ultrasound beam,
all transmitted pulses contain frequencies in a first harmonic band and the second harmonic component of this band, where the polarity of the first harmonic frequency components of said transmitted pulses are equal, and where the second harmonic component of each transmitted pulse has opposite polarity of the second harmonic component of the previously transmitted pulse,
the scattered signal from said pulses are received with an ultrasound transducer with substantially same beam direction and focusing for said pulses, and the received signal from all or some of the transmitted pulses are combined with each other,
so that linear and/or first order nonlinear components in the received signals from the tissue are greatly reduced in the combination of the signals.

4. A method for imaging of ultrasound contrast agents according to claim 3, where the amplitudes of said first and second harmonic frequency components of one transmitted pulse are different from the amplitudes of said first and second harmonic frequency components of another transmitted pulse.

5. A method for imaging of ultrasound contrast agents according to claim 3, where the amplitudes of both said first and said second harmonic frequency components are the same among all said transmitted pulses.

6. A method for imaging of ultrasound contrast agents according to claim 3, where only two ultrasound pulses are transmitted.

7. A method for imaging of ultrasound contrast agents according to claim 3, where the received signals from said pulses are combined through summation of the received signals.

8. A method for imaging of ultrasound contrast agents according to claim 3, where the received signals from said pulses are combined by summing the received signals from the odd number transmitted pulses and subtracting the received signals from the even number transmitted pulses.

9. A method for imaging of ultrasound contrast agents according to claim 3, where the combination of said signals has the form of a linear filter along the pulse number coordinate for each depth range.

10. A method for imaging of ultrasound contrast agent according to claim 3, where in said transmitted pulses the phase of said second harmonic band relative to said first harmonic band is adjusted between 0 and
$$\frac{π}{2}$$
for maximizing the detected signal power from the contrast agent.

11. A method for imaging of ultrasound contrast agent according to claim 1, where in said transmitted pulse(s) the phase of the second harmonic band relative to the first harmonic band is adjusted based on maximized displayed intensity of the detected contrast agent signal in the image.

12. A method for imaging of ultrasound contrast agent according to claim 1, where in said transmitted pulses the phase of the second harmonic band relative to the first harmonic band is automatically adjusted to maximizing the intensity of the detected contrast agent signal.

13. A method for imaging of ultrasound contrast agent according to claim 3, where RF-filtering is applied in addition to a combination of the received signals to further attenuate unwanted tissue components.

14. A method for imaging of ultrasound contrast agent according to claim 3, where the ultrasound beam is swept continuously with a mechanical motion of the transducer, the motion being so slow in relation to the pulse transmit rate that the received signals from two or more transmitted pulses originate from substantially the same region in the tissue.

15. A method for imaging of ultrasound contrast agent according to claim 3, where the beam direction is stepped to form the image, and the beam direction is the same for two or more transmitted pulses that are used to form the detected signal from the contrast agent for said beam direction.

16. A method for imaging of ultrasound contrast agent according to claim 3, where in said transmitted pulse(s) the phase of the second harmonic band relative to the first harmonic band is adjusted based on maximized displayed intensity of the detected contrast agent signal in the image.

17. A method for imaging of ultrasound contrast agent according to claim 3, where in said transmitted pulses the phase of the second harmonic band relative to the first harmonic band is automatically adjusted to maximizing the intensity of the detected contrast agent signal.
